(19) United States
(12) Patent Application Publication
(10) Pub. No.: US 2023/0210796 A1
(43) Pub. Date: Jul. 6, 2023

(54) COMPOSITIONS AND METHODS FOR TREATING HEADACHES

(71) Applicant: TECHNION RESEARCH AND DEVELOPMENT FOUNDATION LIMITED, Haifa (IL)

(72) Inventors: David Meiri, Tzur Moshe (IL); Joshua Aviram, Haifa (IL)

(21) Appl. No.: 18/000,392

(22) PCT Filed: May 31, 2021
(86) PCT No.: PCT/IL2021/050645

§ 371 Date: Nov. 30, 2022

Related U.S. Application Data
(60) Provisional application No. 63/035,778, filed on Jun. 7, 2020.
(60) Provisional application No. 63/032,771, filed on Jun. 1, 2020.

(51) Int. Cl. A61K 31/192; A61P 25/06; A61K 45/06

(52) U.S. Cl. CPC A61K 31/192; A61P 25/06; A61K 45/06

(57) ABSTRACT

The present invention relates to pharmaceutical compositions containing certain phytocannabinoids and their use in treating or preventing headaches, particularly migraine headaches.

### FIELD OF THE INVENTION

The present invention relates to compositions and methods for treating or preventing headaches, particularly migraine headaches.

### BACKGROUND OF THE INVENTION

Chronic migraine constitutes a disabling neurological disorder, affecting around one to two percent of the global population worldwide. It is manifested as recurrent attacks of moderate to severe headache pain typically lasting between 4 and 72 hours. Most migraines are unilateral, have a pulsating quality, are aggravated by routine physical activity, and are associated with nausea and/or sensitivity to light and sound. In migraines with aura, the headache phase is preceded by reversible focal neurological symptoms, often visual or sensory, that usually develop gradually over 5 to 20 minutes and last for 15 migraine days per month (Lars et al. Nat Rev Neurol. 2018, 14(6), 338-350).

Multiple studies have confirmed that release of calcitonin gene-related peptide (CGRP) is increased during acute migraine attacks. In the trigeminal ganglion, CGRP is expressed in C-fibers and its receptor is expressed in Δδ-fibers. These types of fibers are involved in different aspects of pain perception. The trigeminal ganglion is central to the trigeminovascular reflex, which is triggered to protect against vasoconstriction. Triggering of this system in patients with migraines leads to the perception of pain. CGRP is released upon activation of the trigeminal ganglion (Goadsby et al. Ann Neurol. 1988, 23(2), 193-196) and induces migraine-like attack in migraine patients (Lassen et al. Cephalalgia. 2002, 22(1), 54-61). Thus, it is contemplated that CGRP plays an important role in the development of migraine attacks (Ashina et al. Cephalalgia. 2018, 38(2), 353-360).

Current treatments for migraines include triptans, non-steroidal anti-inflammatory drugs (NSAIDs), paracetamol, ergots, opioids, and antiemetics. Preventive treatments include antidepressants, anticonvulsants, and β-blockers. Recently, CGRP receptor antagonists, anti-CGRP antibodies and anti-CGRP receptor antibodies were shown to be effective for migraine pain relief. Although these antagonists and antibodies show promising results in migraine patients, there are risks of long-term CGRP blockade. Therefore, the development of effective drugs that act on this target and do not have long-term effects are highly desirable for the treatment of patients with migraine.

In recent years, the use of medical cannabis (MC) for the treatment of chronic pain has emerged, along with an increase in demand and use by migraine patients. A recent cross-sectional study found that nearly 36% of MC users reported using it to treat headache and migraine (Sexton et al., Cannabis Cannabinoid Res. 2016, 1, 131-138, doi:10.1089/can.2016.0007). An additional survey reported about 50% reduction of migraine and headache severity following inhaled cannabis consumption (Cuttler et al., J. Pain 2019, doi:10.1016/j.jpain.2019.11.001). Nevertheless, good clinical data supporting the beneficial effect of MC on migraine are scarce.

Both clinical and pre-clinical data suggest that abnormality in the endocannabinoids system has a key role in migraine etiology. In patients with chronic migraines, the cerebral spinal fluid (CSF) concentrations of anandamide (AEA) were significantly lower and the concentrations of palmitoylethanolamide (PEA) were significantly higher compared to patients who do not suffer from migraine headaches and controls. Furthermore, reduced levels of AEA degrading enzymes were found in platelets of patients with chronic migraines. In animal models of migraine, administration of AEA diminished hyperalgesic behavior and the plant-derived THC showed anti-migraine effects in rats (Greco et al., J. Headache Pain 2011, 12, 177-183, doi:10.1007/s10194-010-0274-4). Mechanistically, endocannabinoids exert an inhibitory effect on serotonin receptors in vivo (Fan, P. J. Neurophysiol. 1995, 73, 907-910, doi:10.1152/jn.1995.73.2.907), which is shown to modulate pain and emetic responses. Additional in vivo data showed that THC induced an antinociception effect on the periaqueductal gray matter (Lichtman et al., J. Pharmacol. Exp. Ther. 1996; Vol. 276 (2), 585-593), which is believed to be involved in migraine pathophysiology (Haroutounian et al. Clin. J. Pain 2016, 32, 1036-1043, doi:10.1097/AJP.0000000000000364). Nonetheless, these studies do not incorporate all of the complexities of MC treatment.

The cannabis plant contains hundreds of different active components including phytocannabinoids, terpenes and flavonoids. While THC and cannabidiol (CBD) are among the most well-known phytocannabinoids, others are likely to have biological activity as well. The multi-compound effect of cannabis termed “the entourage effect”, suggests that studies examining the role of single-molecule cannabinoids in disease may not necessarily capture the synergies in multi-compound MC treatment. To add to the complexity of MC treatment with multiple compounds, there are hundreds of different cannabis chemovars, each having its own unique chemical composition. Recently, an ESI-LC/MS/MS approach for comprehensive identification and quantification of phytocannabinoids in cannabis has been developed. Over 90 phytocannabinoids, of which approximately 20 were previously unknown, have been identified (Baram et al., Oncotarget. 2019, 10, 4091-4106, doi:10.18632/oncotarget.26983). Quantifying the multitude of phytocannabinoids is the first step in order to obtain better understandings of the therapeutic potential of each cannabis chemovar.

Previous studies on migraines usually regarded cannabis as a single adherent medication (Rhyne, Pharmacother. J. Hum. Pharmacol. Drug Ther. 2016, 36, 505-510, doi:10.1002/phar.1673), and therefore disregard the inherent complexity in MC treatment with differences in over 90 phytocannabinoids between cannabis cultivars (Ben-Shabat et al. Eur. J. Pharmacol. 1998, 353, 23-31, doi:10.1016/S0014-2999(98)00392-6; Hazekamp et al., Drug Test. Anal. 2012, 4, 660-667, doi:10.1002/dta.407).

U.S. 2019/0275270 describes a device for treating a migraine event, said device comprising: a) caffeine; b) a cannabinolic active substance; c) a carrier liquid; and d) a delivery device that produces atomized particles.

U.S. 2009/0232898 describes a pharmaceutical composition for the treatment of migraine comprising: (a) a pharmacologically-effective amount of a triptan or an ergot, or a pharmaceutically-acceptable salt thereof; (b) a pharmacologically-effective amount of an antiemetic compound, or a pharmaceutically-acceptable salt thereof; (c) a bioadhesion and/or a mucoadhesion promoting agent; and (d) carrier particles, wherein (1) active ingredients (a) and (b) are presented in particulate form upon the surfaces of the carrier particles, which carrier particles are larger in size than the particles of the active ingredients; and (2) the bioadhesion and/or mucoadhesion promoting agent is, at least in part, presented on the surfaces of the carrier particles. The antiemetic compound is, inter alia, a cannabinoid.

U.S. 2016/0256411 describes a method of treating a disease state or condition in human with a cannabinoid drug(s) comprising administering a cannabinoid drug(s) in a therapeutically effective amount to treat the disease state or condition, to the back of the neck region of a human patient to provide regional neuro-affective therapy to the human patient. The disease state or condition is, inter alia, headaches (migraine and tension).

WO 2002/064109 describes a pharmaceutical formulation which comprises both the cannabinoids THC and THCV wherein the THCV is present in an amount by weight which is approximately equal to or greater than the amount by weight of THC for use in the treatment of cancer pain or migraine or for stimulation of the appetite. WO 2002/064109 further describes a pharmaceutical formulation which comprises a ratio by weight of THC to CBD or THCV to CBDV of from about 39:1 to about 99:1 for use in the treatment of cancer pain or migraine or for stimulation of appetite.

There remains an unmet need for new treatment modalities of headaches. In particular, there remains an unmet need for compounds and compositions that provide highly effective treatment of migraine headaches and prevent migraine attacks.

### SUMMARY OF THE INVENTION

The present invention provides compositions and methods for treating or preventing headaches. In particular, the compositions comprise at least one cannabinoid denoted CAN001 and CAN002 (cannabigerol monomethyl ether (CBGM)) and use thereof in treating or preventing headaches.

The present invention is based in part on the unexpended findings of two cannabinoids CAN001 and CBGM that were shown to exert beneficial effects in the treatment of patients suffering from migraine attacks. In particular, a cross-sectional study was performed to calculate the total dose of individual phytocannabinoids consumed by migraine patients and explore differences of their dose between subgroups of patients according to their changes in frequency of migraine attacks. Additionally, associations between the change in frequency of migraine attacks to migraine disability severity, sleep quality and timing and migraine analgesics consumption were explored. CAN001 and CBGM, were shown to reduce the frequency of migraine attacks and the negative headache impact while improving the migraine disability status and sleep quality.

According to a first aspect, there is provided a pharmaceutical composition comprising at least one cannabinoid selected from CAN001, CBGM, and pharmaceutically acceptable salts or derivatives thereof, for use in treating or preventing a headache.

In one embodiment, the pharmaceutical composition comprises a single cannabinoid comprising CAN001 or pharmaceutically acceptable salts or derivatives thereof. In another embodiment, the pharmaceutical composition comprises a single cannabinoid comprising CBGM or pharmaceutically acceptable salts or derivatives thereof. In yet another embodiment, the pharmaceutical composition comprises two cannabinoids comprising a combination of CAN001 and CBGM or pharmaceutically acceptable salts or derivatives thereof. In further embodiments, the pharmaceutical composition comprises CAN001 or pharmaceutically acceptable salts or derivatives thereof and at least one additional cannabinoid. In additional embodiments, the pharmaceutical composition comprises CBGM or pharmaceutically acceptable salts or derivatives thereof and at least one additional cannabinoid.

In some embodiments, the pharmaceutical composition comprises CAN001 and CBGM or pharmaceutically acceptable salts or derivatives thereof in weight percent ratios ranging from 1,000:1 to 1:1,000, including all iterations of ratios within the specified range. In other embodiments, the pharmaceutical composition comprises CAN001 and CBGM or pharmaceutically acceptable salts or derivatives thereof in weight percent ratios ranging from 100:1 to 1:100, including all iterations of ratios within the specified range. In further embodiments, the pharmaceutical composition comprises CAN001 and CBGM or pharmaceutically acceptable salts or derivatives thereof in weight percent ratios ranging from 2:1 to 1:100, including all iterations of ratios within the specified range. In additional embodiments, the pharmaceutical composition comprises CAN001 and CBGM or pharmaceutically acceptable salts or derivatives thereof in weight percent ratios ranging from 1:2 to 1:100, including all iterations of ratios within the specified range.

In other embodiments, the pharmaceutical composition comprises at least 0.5% (w/w) of CAN001, and CBGM, and pharmaceutically acceptable salts or derivatives thereof. In yet other embodiments, the pharmaceutical composition comprises at least 1% (w/w) of CAN001, and CBGM, and pharmaceutically acceptable salts or derivatives thereof. In further embodiments, the pharmaceutical composition comprises at least 5% (w/w) of CAN001, and CBGM, and pharmaceutically acceptable salts or derivatives thereof. In additional embodiments, the pharmaceutical composition comprises at least 10% (w/w) of CAN001, and CBGM, and pharmaceutically acceptable salts or derivatives thereof. In specific embodiments, the pharmaceutical composition comprises at least 20% (w/w) of CAN001, and CBGM, and pharmaceutically acceptable salts or derivatives thereof. In particular embodiments, the pharmaceutical composition consists essentially of CAN001, and CBGM, and pharmaceutically acceptable salts or derivatives thereof.

In some embodiments, the pharmaceutical composition further comprises at least one additional cannabinoid selected from the group consisting of tetrahydrocannabinol (THC), THC-C4, tetrahydrocannabivarin (THCV), cannabinol (CBN), cannabigerol (CBG), cannabidiol (CBD), cannabichromene (CBC), and a mixture or combination thereof. Each possibility represents a separate embodiment.

In further embodiments, the pharmaceutical composition further comprises at least one additional cannabinoid selected from the group consisting of tetrahydrocannabinol (THC), THC-C4, tetrahydrocannabivarin (THCV), cannabinol (CBN), cannabigerol (CBG), cannabidiol (CBD), cannabichromene (CBC), cannabidivarinic acid (CBDVA), cannabidiolic acid (CBDA), cannabigerolic acid (CBGA), cannabinolic acid (CBNA), cannabichromenic acid (CBCA), tetrahydrocannabinolic acid (THCA), THCA-C4, cannabicitran, sesquicannabigerol (sesqui-CBG), sesquicannabigerolic acid (sesqui-CBGA), CBGA-C4, CBG-C4, cannabigerovarinic acid (CBGVA), cannabigerivarin (CBGV), cannabigerorcinic acid (CBGOA), cannabigerorcin (CBGO), cannabicyclol (CBL), cannabicyclolic acid (CBLA), tetrahydrocannabivarin carboxylic acid (THCVA), tetrahydrocannabiorcolic acid (THCOA), tetrahydrocannabiorcol (THCO), THCMA, THCM, CBDA-C4, CBD-C4, cannabidiorcolic acid (CBDOA), cannabidiorcol (CBDO), cannabidiolic acid monomethyl ether (CBDMA), cannabidiol monomethylether (CBDM), cannabichromenic acid (CBCA), cannabichromene (CBC), cannabichromevarinic acid (CBCVA), cannabichromevarin (CBCV), cannabiorchromenic acid (CBCOA), cannabiorchromene (CBCO), cannabinolic acid (CBNA), cannabinol-C4 (CBN-C4), cannabivarinic acid (CBNVA), cannabivarin (CBNV), cannabiorcolic acid (CBNOA), cannabiorcol (CBNO), CBNA-8-OH, CBN-8-OH, cannabinol methylether (CBNM), cannabielsoin acid (CBEA), cannabielsoin (CBE), cannabielsoic acid (CBEVA), cannabielsoin (CBEV), cannabinodiolic acid (CBNDA), cannabinodiol (CBND), cannabinodivarinic acid (CBNDVA), (−)-Δ⁸-trans-tetrahydrocannabinol (Δ⁸-THC), cannabitriol-1 (CBT-1), CBT-2, CBT-3, CBTA-1, CBTA-3, cannabitriolvarin (CBTV), CBTV-3, and a mixture or combination thereof. Each possibility represents a separate embodiment.

In certain embodiments, the pharmaceutical composition comprises three cannabinoids comprising THC, CAN001, and CBGM, and pharmaceutically acceptable salts or derivatives thereof. In various embodiments, the pharmaceutical composition comprises CAN001 and at least one cannabinoid selected from THC and CBGM, or pharmaceutically acceptable salts or derivatives thereof. In other embodiments, the pharmaceutical composition comprises three cannabinoids comprising THC, CAN001, and CBGM, and pharmaceutically acceptable salts or derivatives thereof in weight percent ratios ranging from 2,000:1,000:1 to 1:1:1,000, including all iterations of ratios within the specified range. In yet other embodiments, the pharmaceutical composition comprises three cannabinoids comprising THC, CAN001, and CBGM, and pharmaceutically acceptable salts or derivatives thereof in weight percent ratios ranging from 2,000:100:1 to 50:1:100, including all iterations of ratios within the specified range. In additional embodiments, the pharmaceutical composition comprises three cannabinoids comprising THC, CAN001, and CBGM, and pharmaceutically acceptable salts or derivatives thereof in weight percent ratios ranging from 2,000:2:1 to 50:1:100, including all iterations of ratios within the specified range. In some embodiments, the pharmaceutical composition comprises at least 20% (w/w) of THC, CAN001, and CBGM, and pharmaceutically acceptable salts or derivatives thereof. In other embodiments, the pharmaceutical composition comprises at least 25% (w/w) of THC, CAN001, and CBGM, and pharmaceutically acceptable salts or derivatives thereof. In yet other embodiments, the pharmaceutical composition comprises at least 30% (w/w) of THC, CAN001, and CBGM, and pharmaceutically acceptable salts or derivatives thereof. In particular embodiments, the pharmaceutical composition consists essentially of THC, CAN001, and CBGM, and pharmaceutically acceptable salts or derivatives thereof. In yet other embodiments, the pharmaceutical composition comprises less than 1% by weight of CBD.

In some embodiments, the at least one cannabinoid is a natural compound, a synthetic compound, a semi-synthetic compound, or a mixture thereof. Each possibility represents a separate embodiment.

In various embodiments, the pharmaceutical composition further comprises at least one pharmaceutically acceptable excipient. In particular embodiments, the at least one pharmaceutically acceptable excipient is selected from the group consisting of a binder, a filler, a surfactant, an anti-tacking agent, a plasticizer, a lubricant, a glidant, a disintegrant, a diluent, a tonicity enhancing agent, a wetting agent, a mucoadhesive agent, a buffering substance, a colorant, a preservative, and any combination thereof, with each possibility representing a separate embodiment.

In additional embodiments, the pharmaceutical composition is in a dosage form selected from tablet, pill, capsule, strip, pellets, granules, powder, lozenge, sachet, cachet, elixir, suspension, dispersion, emulsion, solution, syrup, aerosol, foam, gel, ointment, lotion, cream, and suppository. Each possibility represents a separate embodiment.

In other embodiments, the pharmaceutical composition is adapted for administration via a route selected from oral, buccal, sublingual, subcutaneous, intratracheal, intrabronchial, intra-alveolar, intraperitoneal, rectal, intravenous, intra-arterial, transdermal, intramuscular, topical, and intranasal. Each possibility represents a separate embodiment.

In particular embodiments, the headache is a migraine headache.

In further embodiments, treating or preventing a headache comprises reducing the occurrence of headaches. In additional embodiments, treating or preventing a headache comprises reducing at least one symptom associated with the headache.

In another aspect, there is provided a method of treating or preventing a headache, the method comprising administering to a subject in need thereof a pharmaceutical composition as disclosed herein. In one embodiment, the subject is a mammal, preferably a human.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C. Clinical differences between responders and non-responders. FIG. 1A: Migraine index disability scale (MIDAS) score: responders n=70 and non-responders n=41; p<0.05; FIG. 1B: Headache impact test (HIT-6) score: responders n=70 and non-responders n=43; p<0.001; FIG. 1C: Pittsburgh sleep quality index (PSQI) score: responders n=62 and non-responders n=34; p<0.05; Response refers to reduction in the frequency of monthly migraine attacks following the initiation of MC treatment (i.e., ≥50%) compared to non-responders (i.e., <50%).

FIG. 2. Cannabinoids relative dose in the most frequently consumed cultivars. Shades on the graph represent the scaled phytocannabinoid concentration variations between cultivars; the numbers in each box represent the concentration (%) of the specific phytocannabinoid within each cultivar; *for each phytocannabinoid, the concentrations of the acid and its neutral counterpart were summed and reported as the total content; Method used: package “pheatmap”, function pheatmap, with the (default): distance measure used in clustering rows “euclidean”, clustering method used is “complete” on z scored data scaled by row; THC, (−)-Δ⁹-trans-tetrahydrocannabinol; CBD, cannabidiol; CBC, cannabichromene; CBG, cannabigerol; CBN, cannabinol; THC-C4, (−)-Δ⁹-trans-tetrahydrocannabinol-C4; THCV, (−)-Δ⁹-trans-tetrahydrocannabivarin.

FIGS. 3A-3C. Phytocannabinoids dose differences between responders and non-responders. FIG. 3A: Medical cannabis (MC) total monthly dose (grams), responders n=45 and non-responders n=23; p=0.97; FIG. 3B: Frequency (%) of patients receiving high (□) and low (▪) monthly doses of ms_373_15c (CAN002), responders n=45 and non-responders n=23; p<0.05; FIG. 3C: Frequency (%) of patients receiving high (□) and low (▪) monthly doses of ms_331_18d (CAN001), responders n=45 and non-responders n=23; p<0.01. Response refers to reduction in frequency of monthly migraine attacks following the initiation of MC treatment (i.e., ≥50%) compared to non-responders (i.e., <50%).

### DETAILED DESCRIPTION OF THE INVENTION

The present invention provides compositions containing CAN001 and/or cannabigerol monomethyl ether (CBGM) phytocannabinoids and use thereof in treating headaches, particularly migraines.

According to certain aspects and embodiments, there is provided a pharmaceutical composition comprising at least one cannabinoid selected from CAN001, CBGM, and pharmaceutically acceptable salts or derivatives thereof, for use in treating or preventing a headache.

According to other aspects and embodiments, there is provided a method of treating or preventing a headache, the method comprising administering to a subject in need thereof a pharmaceutical composition comprising at least one cannabinoid selected from CAN001, CBGM, and pharmaceutically acceptable salts or derivatives thereof.

According to further aspects and embodiments, there is provided the use of at least one cannabinoid selected from CAN001, CBGM, and pharmaceutically acceptable salts or derivatives thereof for the preparation of a medicament for treating or preventing a headache.

Cannabinoids

As used herein and in the appended claims, the pharmaceutical compositions of the present invention comprise at least one cannabinoid selected from CAN001, CBGM, and pharmaceutically acceptable salts or derivatives thereof. The term “derivative” is meant to encompass the phytocannabinoids denoted as CAN001 and CBGM in their neutral form and/or in their acid form. This term also encompasses modified phytocannabinoids containing e.g. an ester moiety, an amide moiety, an enol moiety, as well as different isomeric forms such as an enantiomer, diastereoisomer or tautomer, racemic mixtures, prodrugs, or complexes, either in pure form or in admixture. Each possibility represents a separate embodiment.

In one embodiment, the pharmaceutical composition comprises at least one cannabinoid which is denoted CAN001 or pharmaceutically acceptable salts or derivatives thereof. CAN001 having a molecular weight as determined by Mass Spectra of 331.22790 g/mol is characterized by the following chemical formula C₂₁H₃₂O₃. It is to be understood that the cannabinoid termed CF1 disclosed in WO 2020/230145 while having the same molecular weight and chemical formula as CAN001, corresponds to a different chemical structure due to its different retention time and fragmentation pattern. While it is noted that the molecular weight of 331.22790 g/mol corresponds to the deprotonated form of the neutral phytocannabinoid, variations in the molecular weight from 331.22790 g/mol depending on the form of the phytocannabinoid are contemplated by the present invention. It is to be understood that any reference to CAN001 as used herein encompasses the decarboxylated form, the acid form, or a mixture thereof.

In another embodiment, the pharmaceutical composition comprises at least one cannabinoid which is denoted CAN002 (i.e. cannabigerol monomethyl ether (CBGM)) or pharmaceutically acceptable salts or derivatives thereof. It is to be understood that any reference to CBGM as used herein encompasses the decarboxylated form (CBGM), the acid form cannabigerolic acid monomethyl ether (CBGMA), or a mixture thereof. CBGMA having a molecular weight as determined by Mass Spectra of 373.23840 g/mole (deprotonated) is characterized by the following chemical formula C23H₃₄O₄ and the following chemical structure:

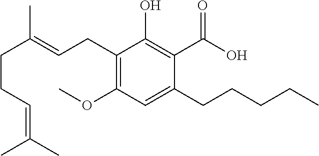 [SMILES: CCCCCc1cc(OC)c(C/C=C(\C)CCC=C(C)C)c(O)c1C(=O)O]

While CBGMA has a molecular weight of 374.5 g/mol, it is clear that the mass of 373.23840 g/mole as determined by LC-MS corresponds to the deprotonated form of the compound.

CAN001 and CBGMA can further be identified by LC/MS/MS as described in Berman et al. (Scientific Reports, 2018, 8, 14280, DOI:10.1038/s41598-018-32651-4), the contents of which are hereby incorporated by reference in their entirety. Briefly, identification of phytocannabinoids can be performed by analyzing the analytical phytocannabinoid standards and cannabis samples in the prepared concentration. Data dependent MS/MS mode is performed using a Thermo Scientific UHPLC system coupled with a Q Exactive™ Hybrid Quadrupole-Orbitrap MS (Thermo Scientific, Bremen, Germany). The chromatographic separation is achieved using a Kinetex C18 core-shell column (2.6 μm, 150 mm×2.1 mm i.d.) with a guard column (0.5 μm depth filter×0.1 mm) (Phenomenex, Torrance, Calif., USA) and a ternary A/B/C multistep gradient (solvent A: 0.1% acetic acid in Milli Q water, solvent B: 0.1% acetic acid in acetonitrile, and solvent C: methanol, all solvents have LC/MS grade). Solvent C is kept constant at 5% throughout the run. The multistep gradient program can be established as follows: initial conditions: 50% B raised to 67% B until 2 min, held at 67% B for 4 min, and then raised to 90% B until 10 min, held at 90% B until 14 min, decreased to 50% B over the next min, and held at 50% B until 20 min for re-equilibration of the system prior to the next injection. A flow rate of 0.3 ml/min is used, the column temperature is 30° C. and the injection volume is 1 μL. MS acquisition is performed with a heated electro spray ionization (HESI-II) ion source operated in negative mode. Source parameters are as follows: sheath gas flow rate, auxiliary gas flow rate, and sweep gas flow rate: 50, 20, and 0 arbitrary units, respectively; capillary temperature: 350° C.; heater temperature: 50° C.; spray voltage: 3.00 kV. The scan range is 150-550 m/z for all acquisition events. MS is operated in full MS1 followed by data dependent MS/MS mode with an NCE of 40. Data acquisition in full MS1 mode is performed at 70,000 resolution, and the AGC target is set to 10⁶ with a maximum IT of 100 ms. Data acquisition in data dependent MS/MS mode is performed at 17,500 resolution, the AGC target is set to 10⁵ with a maximum IT of 50 ms and an isolation window of 4 m/z. Using the aforementioned LC/MS/MS protocol, CAN001 and CBGMA are identified as having retention times of 10.12 min, and 13.15 min, respectively.

All stereoisomers of the phytocannabinoids of the present invention are meant to be included within the scope of the present invention, either in admixture or in pure or substantially pure form. These compounds can have asymmetric center(s). Consequently, the compounds can exist in enantiomeric or diastereomeric forms or in mixtures thereof. The present invention contemplates the use of any racemates (i.e. mixtures containing equal amounts of each enantiomers), enantiomerically enriched mixtures (i.e., mixtures enriched in one enantiomer), pure enantiomers or diastereomers, or any mixtures thereof. In addition, the compounds may contain one or more double bonds. The present invention intends to encompass all structural and geometrical isomers including cis, trans, E and Z isomers, independently at each occurrence.

One or more of the phytocannabinoid compounds of the invention, may be present as a salt. The term “salt” encompasses both basic and acid addition salts which may be formed by standard acid-base reactions. Such acids, suitable for forming acid addition salts include, but are not limited to, hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and the like. Each possibility represents a separate embodiment.

Suitable base addition salts include salts having a counterion chosen from the alkali and alkaline earth metals (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. Each possibility represents a separate embodiment. Other cations encompassed by the present invention include, but are not limited to, the protonated form of procaine, quinine and N-methylglucosamine, and the protonated forms of basic amino acids such as glycine, ornithine, histidine, phenylglycine, lysine and arginine. Each possibility represents a separate embodiment of the present invention. Furthermore, any zwitterionic form of the phytocannabinoid compounds is also contemplated.

The present invention may further include solvates of phytocannabinoids of the present invention. “Solvate” as used herein refers to a physical association of a compound with one or more solvent molecules. This physical association involves varying degrees of ionic and covalent bonding, including hydrogen bonding. In certain instances, the solvate will be capable of isolation. “Solvate” encompasses both solution-phase and isolatable solvates. Non-limiting examples of suitable solvates include ethanolates, methanolates, and the like. Each possibility represents a separate embodiment. “Hydrate” is a solvate in which the solvent molecule is water.

The present invention also includes polymorphs of phytocannabinoid compounds. The term “polymorph” refers to a particular crystalline state of a substance, which can be characterized by particular physical properties such as X-ray diffraction, IR and Raman spectra, melting point, and the like.

According to the principles of the present invention, the pharmaceutical composition includes a single cannabinoid selected from CAN001 and CBGM or pharmaceutically acceptable salts and derivatives thereof. In other embodiments, the pharmaceutical composition includes a plurality of cannabinoids, for example 2, 3, 4, 5, 6, 7, 8, 9, 10, or more cannabinoids. Each possibility represents a separate embodiment. In yet other embodiments, the pharmaceutical composition includes CAN001 or pharmaceutically acceptable salts and derivatives thereof and at least one additional cannabinoid which may be CBGM or a different cannabinoid. In further embodiments, the pharmaceutical composition includes CBGM or pharmaceutically acceptable salts and derivatives thereof and at least one additional cannabinoid which may be CAN001 or a different cannabinoid. In additional embodiments, the pharmaceutical composition includes a combination of CAN001 and CBGM or pharmaceutically acceptable salts and derivatives thereof. The weight ratio of CAN001 and CBGM or pharmaceutically acceptable salts or derivatives thereof, when used in combination, is typically in the range of from 1,000:1 to 1:1,000, for example 100:1 to 1:100, 2:1 to 1:100, or 1:2 to 1:100 including all iterations of ratios within the specified ranges. Exemplary ratios include, but are not limited to, about 1,000:1, about 750:1, about 500:1, about 250:1, about 100:1, about 50:1, about 25:1, about 10:1, about 5:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, about 1:100, about 1:250, about 1:500, about 1:750, or about 1:1,000. Each possibility represents a separate embodiment of the present invention. In some embodiments, CAN001, CBGM, and pharmaceutically acceptable salts or derivatives thereof constitute at least 0.5%, at least 1%, at least 5%, at least 10%, or at least 20% (w/w) of the total weight of the pharmaceutical composition. Each possibility represents a separate embodiment. Exemplary weight percent ranges of CAN001, CBGM, and pharmaceutically acceptable salts or derivatives thereof within the composition include, but are not limited to, 0.5% to 100%, 1% to 50%, 2% to 40%, or 3% to 30%, with each possibility representing a separate embodiment. In certain embodiments, CAN001, CBGM, and pharmaceutically acceptable salts or derivatives thereof are the only active ingredients in the pharmaceutical composition of the present invention. In specific embodiments, the pharmaceutical composition consists essentially of CAN001, CBGM, and pharmaceutically acceptable salts or derivatives thereof.

Within the scope of the present invention is the inclusion of one or more additional cannabinoids in the pharmaceutical composition including, but not limited to, tetrahydrocannabinol (THC), THC-C4, tetrahydrocannabivarin (THCV), cannabinol (CBN), cannabigerol (CBG), cannabidiol (CBD), cannabichromene (CBC), and the like. Each possibility represents a separate embodiment.

According to certain aspects and embodiments, the pharmaceutical composition may further include one or more additional cannabinoids such as, but not limited to, tetrahydrocannabinol (THC), THC-C4, tetrahydrocannabivarin (THCV), cannabinol (CBN), cannabigerol (CBG), cannabidiol (CBD), cannabichromene (CBC), cannabidivarinic acid (CBDVA), cannabidiolic acid (CBDA), cannabigerolic acid (CBGA), cannabinolic acid (CBNA), cannabichromenic acid (CBCA), tetrahydrocannabinolic acid (THCA), THCA-C4, cannabicitran, sesquicannabigerol (sesqui-CBG), sesquicannabigerolic acid (sesqui-CBGA), CBGA-C4, CB G-C4, cannabigerovarinic acid (CBGVA), cannabigerivarin (CBGV), cannabigerorcinic acid (CBGOA), cannabigerorcin (CBGO), cannabicyclol (CBL), cannabicyclolic acid (CBLA), tetrahydrocannabivarin carboxylic acid (THCVA), tetrahydrocannabiorcolic acid (THCOA), tetrahydrocannabiorcol (THCO), THCMA, THCM, CBDA-C4, CBD-C4, cannabidiorcolic acid (CBDOA), cannabidiorcol (CBDO), cannabidiolic acid monomethyl ether (CBDMA), cannabidiol monomethylether (CBDM), cannabichromenic acid (CBCA), cannabichromene (CBC), cannabichromevarinic acid (CBCVA), cannabichromevarin (CBCV), cannabiorchromenic acid (CBCOA), cannabiorchromene (CBCO), cannabinolic acid (CBNA), cannabinol-C4 (CBN-C4), cannabivarinic acid (CBNVA), cannabivarin (CBNV), cannabiorcolic acid (CBNOA), cannabiorcol (CBNO), CBNA-8-OH, CBN-8-OH, cannabinol methylether (CBNM), cannabielsoin acid (CBEA), cannabielsoin (CBE), cannabielsoic acid (CBEVA), cannabielsoin (CBEV), cannabinodiolic acid (CBNDA), cannabinodiol (CBND), cannabinodivarinic acid (CBNDVA), (−)-Δ⁸-trans-tetrahydrocannabinol (Δ⁸-THC), cannabitriol-1 (CBT-1), CBT-2, CBT-3, CBTA-1, CBTA-3, cannabitriolvarin (CBTV), CBTV-3, and the like. Each possibility represents a separate embodiment.

In certain embodiments, the pharmaceutical composition comprises three cannabinoids comprising a combination of THC, CAN001, and CBGM, or pharmaceutically acceptable salts or derivatives thereof. In accordance with these embodiments, the weight percent ratios of THC, CAN001, and CBGM are in the range of from 2,000:1,000:1 to 1:1:1,000, including all iterations of ratios within the specified range. Exemplary ratios include, but are not limited to, about 2,000:1,000:1, about 1,500:1,000:1, about 1,000:1,000:1, about 500:1,000:1, about 100:1,000:1, about 50:1,000:1, about 1:1,000:1, about 2,000:750:1, about 1,500:750:1, about 1,000:750:1, about 500:750:1, about 100:750:1, about 50:750:1, about 1:750:1, about 2,000:500:1, about 1,500:500:1, about 1,000:500:1, about 500:500:1, about 100:500:1, about 50:500:1, about 1:500:1, about 2,000:250:1, about 1,500:250:1, about 1,000:250:1, about 500:250:1, about 100:250:1, about 50:250:1, about 1:250:1, about 2,000:100:1, about 1,500:100:1, about 1,000:100:1, about 500:100:1, about 100:100:1, about 50:100:1, about 1:100:1, about 2,000:50:1, about 1,500:50:1, about 1,000:50:1, about 500:50:1, about 100:50:1, about 50:50:1, about 1:50:1, about 2,000:25:1, about 1,500:25:1, about 1,000:25:1, about 500:25:1, about 100:25:1, about 50:25:1, about 1:25:1, about 2,000:10:1, about 1,500:10:1, about 1,000:10:1, about 500:10:1, about 100:10:1, about 50:10:1, about 1:10:1, about 2,000:5:1, about 1,500:5:1, about 1,000:5:1, about 500:5:1, about 100:5:1, about 50:5:1, about 1:5:1, about 2,000:2:1, about 1,500:2:1, about 1,000:2:1, about 500:2:1, about 100:2:1, about 50:2:1, about 1:2:1, about 2,000:1:1, about 1,500:1:1, about 1,000:1:1, about 500:1:1, about 100:1:1, about 50:1:1, about 1:1:1, about 2,000:1:2, about 1,500:1:2, about 1,000:1:2, about 500:1:2, about 100:1:2, about 50:1:2, about 1:1:2, about 2,000:1:5, about 1,500:1:5, about 1,000:1:5, about 500:1:5, about 100:1:5, about 50:1:5, about 1:1:5, about 2,000:1:10, about 1,500:1:10, about 1,000:1:10, about 500:1:10, about 100:1:10, about 50:1:10, about 1:1:10, about 2,000:1:25, about 1,500:1:25, about 1,000:1:25, about 500:1:25, about 100:1:25, about 50:1:25, about 1:1:25, about 2,000:1:50, about 1,500:1:50, about 1,000:1:50, about 500:1:50, about 100:1:50, about 50:1:50, about 1:1:50, about 2,000:1:75, about 1,500:1:75, about 1,000:1:75, about 500:1:75, about 100:1:75, about 50:1:75, about 1:1:75, about 2,000:1:100, about 1,500:1:100, about 1,000:1:100, about 500:1:100, about 100:1:100, about 50:1:100, about 1:1:100, about 2,000:1:250, about 1,500:1:250, about 1,000:1:250, about 500:1:250, about 100:1:250, about 50:1:250, about 1:1:250, about 2,000:1:500, about 1,500:1:500, about 1,000:1:500, about 500:1:500, about 100:1:500, about 50:1:500, about 1:1:500, about 2,000:1:750, about 1,500:1:750, about 1,000:1:750, about 500:1:750, about 100:1:750, about 50:1:750, about 1:1:750, about 2,000:1:1,000, about 1,500:1:1,000, about 1,000:1:1,000, about 500:1:1,000, about 100:1:1,000, about 50:1:1,000, or about 1:1:1,000. Each possibility represents a separate embodiment.

In some embodiments, THC, CAN001, and CBGM, or pharmaceutically acceptable salts or derivatives thereof constitute at least 20%, at least 25%, or at least 30% by weight of the total weight of the pharmaceutical composition. Each possibility represents a separate embodiment. Exemplary weight percent ranges of THC, CAN001, and CBGM, or pharmaceutically acceptable salts or derivatives thereof within the composition include, but are not limited to, 20% to 100%, 20% to 70%, 20% to 60%, 20% to 50%, or 25% to 50%, with each possibility representing a separate embodiment. In certain embodiments, THC, CAN001, and CBGM, or pharmaceutically acceptable salts or derivatives thereof are the only active ingredients in the pharmaceutical composition of the present invention. In specific embodiments, the pharmaceutical composition consists essentially of THC, CAN001, and CBGM, and pharmaceutically acceptable salts or derivatives thereof. In further embodiments, the pharmaceutical composition comprises less than 1% by weight of CBD.

The cannabinoids comprised in the pharmaceutical composition of the present invention may be of a natural source, a synthetic source, a semi-synthetic source, or a mixture thereof. Each possibility represents a separate embodiment. Natural cannabinoids can be obtained from plant tissue, for example the inflorescence, particularly the epidermal hairs called glandular trichomes that are highly abundant on female inflorescences, of any species of the family Cannabaceae, for example Cannabis sativa, Cannabis indica, Cannabis ruderalis, and a mixture or combination thereof. Each possibility represents a separate embodiment. Extraction and purification can be performed as is known in the art, for example by suspending the plant tissue in an appropriate solvent e.g. ethanol or using supercritical CO₂ extraction method followed by purification using winterization, distillation, and the like. Synthetic or semi-synthetic cannabinoids can be obtained using methods known to a skilled artisan, for example by contacting an appropriate substrate with one of cannabinoid synthase enzymes.

### Pharmaceutical Compositions

According to the principles of the present invention, the pharmaceutical composition may further comprise at least one pharmaceutically acceptable excipient. Suitable pharmaceutically acceptable excipients within the scope of the present invention include, but are not limited to, a binder, a filler, a surfactant, an anti-tacking agent, a plasticizer, a lubricant, a glidant, a disintegrant, a diluent, a tonicity enhancing agent, a wetting agent, a mucoadhesive agent, a buffering substance, a colorant, a preservative, and any combination thereof, with each possibility representing a separate embodiment.

Suitable binders include, but are not limited to, polyvinyl pyrrolidone, copovidone, hydroxypropyl methylcellulose, hydroxypropyl cellulose, starch, gelatin, or sugars. Each possibility represents a separate embodiment. Sugars include sucrose, dextrose, molasses, and lactose, with each possibility representing a separate embodiment.

Suitable fillers include, but are not limited to, sugars such as lactose, sucrose, mannitol or sorbitol and derivatives therefore (e.g. amino sugars), ethyl cellulose, microcrystalline cellulose, silicified microcrystalline cellulose and the like, with each possibility representing a separate embodiment.

Suitable surfactants within the scope of the present disclosure include, but are not limited to, non-ionic, anionic or cationic surfactants. Typically, surfactants have one lipophilic and one hydrophilic group in the molecule. The surfactant may optionally comprise one or more of soaps, detergents, emulsifiers, dispersing and wetting agents. More specifically, surfactants may optionally comprise, for example, one or more of polysorbate, stearyl triethanolamine, sodium lauryl sulfate, sodium taurocholate, lauryl amino propionic acid, lecithin, benzalkonium chloride, benzethonium chloride, and glycerin monostearate; and hydrophilic polymers such as polyvinyl alcohol, polyvinyl pyrrolidone, carboxy methylcellulose sodium, methylcellulose, hydoxymethyl cellulose, hydroxyethyl cellulose, and hydroxypropyl cellulose among others, with each possibility representing a separate embodiment.

Suitable anti-tacking agents include, but are not limited to, magnesium stearate, calcium stearate, stearic acid, talc, mica, colloidal silicon and the like among others, with each possibility representing a separate embodiment.

Suitable plasticizers include, but are not limited to, cetyl alcohol, dibutyl sebacate, polyethylene glycol, polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol among others, with each possibility representing a separate embodiment.

Suitable lubricants include, but are not limited to, sodium stearyl fumarate, stearic acid, PEG, or stearates, such as magnesium stearate, with each possibility representing a separate embodiment.

A suitable glidant is e.g., colloidal silicon dioxide.

Suitable disintegrants include, but are not limited to, crospovidone, croscarmellose sodium, a sugar alcohol, a cellulose derivative, cross-linked derivatives of starch (e.g. sodium starch glycolate), pregelatinized starch, crosslinked sodium carboxymethyl cellulose, low substituted hydroxypropyl cellulose and any combination or mixture thereof, with each possibility representing a separate embodiment. Additional disintegrants include, but are not limited to, silicates, carbonates, polyoxyethylene sorbitan fatty acid esters, stearic monoglyceride, guar gum, and lactose. Suitable sugar alcohols include, but are not limited to, mannitol, sorbitol, maltitol, xylitol, and any combination or mixture thereof. Additional sugar alcohols include, but are not limited to, arabitol, isomalt, erythritol, glycerol, lactitol, and mixtures thereof. Suitable cellulose derivatives include, but are not limited to, methylcellulose, cross-linked carboxylic methylcelluloses, microcrystalline cellulose and any combination or mixture thereof. Each possibility represents a separate embodiment.

Suitable diluents include, but are not limited to, dicalcium phosphate dihydrate, sugars, lactose, calcium phosphate, cellulose, kaolin, mannitol, sodium chloride, and dry starch, with each possibility representing a separate embodiment.

Suitable tonicity enhancing agents include, but are not limited to, ionic and non-ionic agents. For example, ionic compounds include, but are not limited to, alkali metal or alkaline earth metal halides, such as, for example, CaCl₂, KBr, KCl, LiCl, NaI, NaBr, and NaCl. Each possibility represents a separate embodiment. Non-ionic tonicity enhancing agents are, for example, urea, glycerol, sorbitol, mannitol, propylene glycol, and dextrose, with each possibility representing a separate embodiment.

Suitable wetting agents include, but are not limited to, glycerin, starches, and the like. Each possibility represents a separate embodiment.

Suitable mucoadhesive agents include, but are not limited to, cellulose derivatives such as hydroxypropylmethyl cellulose (HPMC), hydroxyethyl cellulose (HEC), hydroxypropyl cellulose (HPC), methyl cellulose, ethyl hydroxyethyl cellulose, carboxymethyl cellulose and sodium carboxymethyl cellulose (NaCMC); starch derivatives such as moderately cross-linked starch; acrylic polymers such as carbomer and its derivatives (polycarbophyl); polyethylene oxide (PEO); chitosan (poly-(D-glucosamine)); natural polymers such as gelatin, sodium alginate, pectin; scleroglucan; xanthan gum; guar gum; poly co-(methylvinyl ether/maleic anhydride); microcrystalline cellulose; and croscarmellose. Each possibility represents a separate embodiment.

Suitable buffering substances include, but are not limited to, acidic buffering agents such as short chain fatty acids, citric acid, acetic acid, hydrochloric acid, sulfuric acid, and fumaric acid; and basic buffering agents such as tris, sodium carbonate, sodium bicarbonate, sodium hydroxide, potassium hydroxide, and magnesium hydroxide, with each possibility representing a separate embodiment.

Suitable colorants include, but are not limited to, alumina (dried aluminum hydroxide), annatto extract, calcium carbonate, canthaxanthin, caramel, β-carotene, cochineal extract, carmine, potassium sodium copper chlorophyllin (chlorophyll in-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide, with each possibility representing a separate embodiment.

Suitable preservatives include, but are not limited to, quaternary ammonium salts such as benzalkonium chloride, benzoxonium chloride or polymeric quaternary ammonium salts, alkyl-mercury salts of thiosalicylic acid, such as, for example, thiomersal, phenylmercuric nitrate, phenyl mercuric acetate or phenylmercuric borate, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sorbic acid or ascorbic acid, with each possibility representing a separate embodiment.

Additional excipients that may be incorporated in the compositions of the present invention include agents that control release of the active ingredient(s) such as, but not limited to, polyacrylic copolymer (e.g. Carbopol 934). In one embodiment, the composition is devoid of caffeine. In another embodiment, the composition is devoid of triptan. In yet another embodiment, the composition is devoid of ergot.

According to the principles of the present invention, the pharmaceutical composition is in a dosage form selected from tablet, pill, capsule (soft or hard), strip, pellets, granules, powder, lozenge, sachet, cachet, elixir, suspension, dispersion, emulsion, solution, syrup, aerosol, foam, gel, ointment, lotion, cream, and suppository. Each possibility represents a separate embodiment.

The pharmaceutical composition is formulated for administration via a route selected from oral, buccal, sublingual, subcutaneous, intratracheal, intrabronchial, intra-alveolar, intraperitoneal, rectal, intravenous, intra-arterial, transdermal, intramuscular, topical, and intranasal. Each possibility represents a separate embodiment. In one embodiment, administration is performed to a region that does not include the back of the neck.

Provided herein are pharmaceutical compositions that exhibit release profiles that comprise all possible modes of release profiles including, but not limited to, immediate release (IR), or modified release such as delayed release (DR), sustained release (SR) and extended release (XR) formulations. Each possibility represents a separate embodiment.

Methods of Use

According to the principles of the present invention, the pharmaceutical composition disclosed herein is useful in treating or preventing headaches. In accordance with these embodiments, the present invention provides a method of treating or preventing a headache, the method comprising administering to a subject in need thereof a pharmaceutical composition comprising at least one cannabinoid selected from CAN001, CBGM, and pharmaceutically acceptable salts or derivatives thereof. Each possibility represents a separate embodiment. In other embodiments, the present invention provides the use of at least one cannabinoid selected from CAN001, CBGM, and pharmaceutically acceptable salts or derivatives thereof for the preparation of a medicament for treating or preventing a headache. Each possibility represents a separate embodiment. The term “treating” as used herein refers to the reduction in the occurrence of headaches (i.e. headache frequency) as well as the alleviation of the various symptoms associated with the headache. “Alleviation” as used herein refers to some reduction, significant reduction, near total reduction, and total reduction of one or more symptoms associated with the headache. Additionally, the term “treating” as used herein refers to an improvement in functioning during a headache attack including improvement in sleep quality. The term “treating” as used herein may further refer to reduction in use of conventional treatments including in particular opioids administration. The term “preventing” as used herein refers to the complete or partial prophylaxis of the headache or a symptom associated therewith or the reduction in frequency with which it occurs.

Within the scope of the present invention is the treatment or prevention of migraine headaches. Migraine headaches are recurrent headaches that may be unilateral or bilateral. Migraine headaches may occur with or without a prodrome. The aura of a migraine may consist of neurologic symptoms, such as dizziness, tinnitus, scotomas, photophobia, or visual scintillations (e.g., bright zigzag lines).

The administration regimen can be determined by a skilled artisan depending on various parameters including the patient age, weight etc. as well as the type of headache and the severity and frequency of the headache. The amount of the cannabinoids to be administered in order to confer effective treatment can be determined by standard clinical techniques. In addition, in vitro assays, in vivo assays and ex-vivo assays may optionally be employed to help identify optimal dose ranges. The precise dose to be employed also depends on the route of administration and should be decided according to the judgment of the practitioner and each patient's circumstances. Typically, doses in the range of 0.001 to 1,000 mg/kg of body weight, for example 0.01 mg/kg to 100 mg/kg, 0.1 mg/kg to 100 mg/kg, 1 mg/kg to 100 mg/kg, 10 mg/kg to 75 mg/kg, etc. may be used. Each possibility represents a separate embodiment. Exemplary, non-limiting doses include about 0.001 mg/kg, about 0.01 mg/kg, about 0.1 mg/kg, about 0.5 mg/kg, about 1 mg/kg, about 5 mg/kg, about 10 mg/kg, about 20 mg/kg, about 50 mg/kg, about 60 mg/kg, about 75 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1,000 mg/kg of body weight, with each possibility representing a separate embodiment. Effective doses may be extrapolated from dose-response curves derived from in vitro, animal or ex-vivo model test bioassays or systems. Typical fixed doses include, but not limited to, 0.1 mg, 0.5 mg, 1 mg, 2.5 mg, 5 mg, 10 mg, 20 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1,000 mg, with each possibility representing a separate embodiment.

The administration schedule includes once-daily, twice-daily, thrice-daily, once-weekly, twice-weekly, thrice-weekly, once-monthly, twice-monthly, thrice-monthly, or any other administration schedule known to those of skill in the art. Each possibility represents a separate embodiment. The administration can be continuous, i.e., every day, or intermittent. The terms “intermittent” or “intermittently” as used herein refer to stopping and starting at either regular or irregular intervals. For example, intermittent administration can be administration in one to six days per week or it may mean administration in cycles (e.g. daily administration for two to eight consecutive weeks, then a rest period with no administration for up to one week) or it may mean administration on alternate days.

Unless otherwise defined, all technical and scientific terms used herein have the same meaning as commonly understood by one of ordinary skill in the art to which the invention pertains. In case of conflict, the specification, including definitions, will take precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

As used herein, when a numerical value is preceded by the term “about”, the term “about” is intended to indicate ±10%.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

Participants were migraine patients licensed for MC treatment. Data included self-reported questionnaires and MC treatment features. Patients were retrospectively classified as responders vs. non-responders (≥50% vs. <50% decrease in monthly migraine attacks' frequency following MC treatment initiation, respectively). Comparative statistics evaluated differences between these two subgroups. 145 patients (97 females, 67%) with a median MC treatment duration of three years were analyzed. Compared to non-responders, responders (n=89, 61%) reported lower current migraine disability and lower negative impact and lower rates of opioid and triptans consumption. Subgroup analysis demonstrated that responders consumed higher doses of the phytocannabinoid ms_373_15c (“CAN002, or CBGM”) and lower doses of the phytocannabinoid ms_331_18d (“CAN001”) [3.40 95% CI (1.10 to 12.00); p<0.01 and 0.22 95% CI (0.05-0.72); p<0.05, respectively]. These findings indicate that MC treatment results in long-term reduction of migraine frequency in over 60% of treated patients and is associated with less disability and lower anti-migraine medications intake.

### Materials and Methods

### Subjects

Patients, ages >18 years who were diagnosed with migraine by their physician and had standing MC license for the treatment of any approved condition were eligible to participate in the study.

### Study Procedure

The study was approved prior to data collection by the Institutional Ethics Committee of the Technion, Institute of Technology, Haifa, Israel (#011-2016). The contact information for this cross-sectional study was collected from an existing database of Israeli patients with a preexisting MC license for various indications (n=3,218). Patients who reported being diagnosed with migraine (n=423, 13%), and who signed the electronic consent form and confirmed that migraine was diagnosed by a physician, were eligible to complete the questionnaires. Simultaneously, phytocannabinoids of most clinically administered chemovars of all approved cultivators in Israel were analyzed by LC-MS. The chemical analyses were performed on inflorescence cultivars which were received from the cultivators (and not from the patients). In an attempt to diminish the variability between the cultivars that were analyzed and those utilized by the patients, only phytocannabinoids that were consumed with minimum average concentrations of 0.1 grams per month were analyzed. The individual phytocannabinoid monthly dose was calculated for each patient.

### Study Questionnaires

Data collection was carried out online by secure survey technology Qualtrics® (Provo, Utah, version 12018) (Landshaft et al., The Updated Green Book (May 2019): The Official Guide to Clinical Care in Medical Cannabis 2017). Questionnaires consisted of demographic information including age, gender, BMI and MC treatment duration (years). Data on migraine characteristics included the number of migraine days in the last month and the month prior to MC treatment initiation, age of migraine initiation, average current duration of migraine attack (hours) and the presence of aura, nausea/vomiting, photo/phonophobia, uni/bilateral or aggravation in activity of the migraine attack. Information on the analgesics and the specific migraine abortive/preventive medications were collected. Validated questionnaires included the migraine index disability scale (MIDAS) (Qualtrics, L.L.C., Version 12018. Provo, Utah, USA, Retrieved from http//www.qualtrics.com 2015), the headache impact test (HIT-6) (Stewart et al., J. Development and testing of the Migraine Disability Assessment (MIDAS) Questionnaire to assess headache-related disability, Neurology 2001, 56), and the Pittsburgh sleep quality index (PSQI) (Yang et al. Validation of the Headache Impact Test (HIT-6™) across episodic and chronic migraine, Cephalalgia 2011, 31, 357-367, doi: 10.1177/0333102410379890). Additionally, MC treatment characteristics included administration mode, cultivator brand, cultivar name, total monthly dose (grams), monthly dose of each specific cultivar names (grams) and related adverse effects (AEs).

### Phytocannabinoid Profiling of Cannabis Chemovars

Air-dried medical cannabis chemovars were obtained from several Israeli medical cannabis cultivators. Reagents, analytical standards and general methodologies for phytocannabinoid extraction and analysis from cannabis were conducted according to previously published methods (Baram et al., Oncotarget 2019, 10, 4091-4106, doi:10.18632/oncotarget.26983; Berman et al. Scientific Reports 2018, 8, 14280, DOI:10.1038/s41598-018-32651-4, the contents of which are hereby incorporated by reference in their entirety).

Briefly, for phytocannabinoid extraction, 100 mg of ground cannabis flowers were accurately weighed and extracted with 1 mL ethanol. Samples were agitated in an orbital shaker at 25° C. for 15 minutes, and then centrifuged at 4,200 rpm. A fraction of the supernatant was collected and filtered through a 0.22 μm PTFE syringe filter and diluted in the ratios of 1:9, 1:99 and 1:999 v/v cannabis extract to ethanol. Phytocannabinoid analyzes were performed using a Thermo Scientific ultra-high-performance liquid chromatography (UHPLC) system coupled with a Q Exactive™ Focus Hybrid Quadrupole Orbitrap mass spectrometer (MS, Thermo Scientific, Bremen, Germany). The chromatographic conditions were according to Baram et al. (Oncotarget 2019, 10, 4091-4106, doi:10.18632/oncotarget.26983) and Berman et al. (Scientific Reports 2018, 8, 14280, DOI: 10.1038/s41598-018-32651-4). Identification and absolute quantification of phytocannabinoids was performed by external calibrations. Compounds for which there were no commercially available analytical standards were semi-quantified. For each phytocannabinoid, the concentrations of the acid and its neutral counterpart were summed and reported as the total content. For example, the concentration of total Δ⁹-THC was calculated as Total Δ⁹-THC=Δ⁹-THCA×0.877+Δ⁹-THC where 0.877 is the molar ratio between the two compounds which corrected for a change in the mass of Δ⁹-THCA as a result of decarboxylation. For compounds with no absolute identification, neutral or acid concentrations were utilized. Thus, the concentrations of CAN001 were based on it being in a neutral form while the concentrations of CAN002 were based on CBGMA.

### Statistical Analysis

R software (V.1.1.463) with tidyverse, pheatmap and atable packages (Shochat et al., Isr. Med. Assoc. J. 2007, 9, 853-856; Wickham, Tidyverse: Easily install and load “Tidyverse” packages. Version 1.3.0. Compr. R Arch. Netw. 2019; Kolde, R. R package pheatmap: Pretty Heatmaps. Version 1.0.8. Comprehensive R Archive Network (CRAN) 2015) were used to analyze differences in outcome measures by pearson chi-square for categorical measures and Kruskal-Wallis rank sum test for numeric measures. For effect size (i.e., odds ratio, OR) and confidence interval (CI) Cohen's d test was utilized. As customary in recent migraine clinical trials (Strobel et al., R package atable: Create Tables for Reporting Clinical Trials. Version 0.1.5. Comprehensive R Archive Network (CRAN) 2019), the primary outcome of this study was the clinically significant reduction in migraine attacks monthly frequency following the initiation of MC treatment (i.e., ≥50%; responders) compared to non-responders (i.e., <50%). Shapiro-Wilk test of normality demonstrated non-normal distribution for all measures. Thus, data are presented as median and quartiles 25 and 75 (Q1-Q3). Differences were considered significant at the p<0.05 level. Incidences are presented as number and percentage of patients.

### Results

### Subjects

A patient reported outcomes database of Israeli patients with a preexisting MC license for various MCU approved indications (n=3,218) was established. Specific information on this database was described in Hergenrather et al. (Rambam Maimonides Med. J. 2020, 11, 1-14, doi:10.5041/RMMJ.10384). In this database population, 423 (13%) patients reported having a diagnosis of migraine. These patients' main reason for MC license was chronic non-cancer pain (81%), but also cancer related disorders (9%), post-traumatic stress disorder (7%), gastrointestinal disorders (2%) and neurological disorders (1%). 231 (54%) of these patients participated in the current study.

145 patients reported on the monthly migraine attacks frequency before and after MC treatment initiation. The information of these patients was analyzed and is reported herein. The sample consisted of a majority of females (n=97, 67%) ages 34-54 (median age of 45). These patients were treated with MC for over a year [3 (2.4-4.6) years], with a range of MC treatment from one year to 12 years (Table 1). Notably, no significant differences were found between responders and non-responders in the demographic and MC treatment measures.

TABLE 1
Demographic and MC treatment characteristics.

| | | | | | |
|---|---|---|---|---|---|
| | Non- | | | | |
| | responders | Responders | Total | Statistic | Effect size |
| Measure | N = 56 | N = 89 | N = 145 | (p) | (CI) |

Number of patients (%)

| | | | | | |
|---|---|---|---|---|---|
| Gender | | | | | |
| Female | 35 | 62 | 97 | 0.51 | 0.73 |
| | (62) | (70) | (67) | (0.48) | (0.34-1.6) |
| Male | 21 | 27 | 48 | | |
| | (38) | (30) | (33) | | |
| Missing N | 0 | 0 | 0 | | |

Median (IQR)

| | | | | | |
|---|---|---|---|---|---|
| Age | 46 | 44 | 45 | 0.08 | −0.02 |
| (years) | (35-54) | (34-54) | (34-54) | (0.96) | (−0.37-0.32) |
| Missing N | 3 | 3 | 6 | | |
| BMI | 25 | 25 | 25 | 0.13 | 0.05 |
| | (22-27) | (22-28) | (22-27) | (0.64) | (−0.28-0.39) |
| Missing N | 0 | 1 | 1 | | |
| MC treatment | 3.5 | 3 | 3 | 0.22 | 0.46 |
| duration (years) | (2.8-5.2) | (2-4) | (2.4-4.6) | (0.09) | (0.10-0.81) |
| Missing N | 4 | 5 | 9 | | |
| MC | | | | | |
| administration mode | | | | | |
| Inflorescence | 40 | 72 | 112 | 2.4 | 0.13 |
| | (71) | (81) | (77) | (0.31) | (0-0.29) |
| Oil extract | 7 | 5 | 12 | | |
| | (12) | (6) | (8) | | |
| Combination^{#} | 7 | 12 | 19 | | |
| | (12) | (13) | (13) | | |
| Missing N | 2 | 0 | | | |
| Inflorescence | | | | | |
| administration mode* | | | | | |
| Pure MC cigarettes | 22 | 33 | 55 | 0.04 | 1.10 |
| | (39) | (37) | (38) | (0.84) | (0.54-2.40) |
| MC cigarettes | 17 | 30 | 47 | 0.01 | 0.89 |
| mixed with tobacco | (30) | (34) | (32) | (0.89) | (0.40-1.90) |
| Bhang | 3 | 11 | 14 | 1.1 | 0.41 |
| | (5) | (12) | (10) | (0.29) | (0.07-1.70) |
| Electronic vaporizer | 14 | 15 | 29 | 1.1 | 0.59 |
| | (25) | (17) | (20) | (0.29) | (0.24-1.50) |
| Manual vaporizer | 5 | 20 | 25 | 3.3 | 2.9 |
| | (9) | (22) | (17) | (0.06) | (0.96-10.00) |
| Missing N | 2 | 1 | | | |
| Oil extract | | | | | |
| administration mode* | | | | | |
| Sublingual | 13 | 13 | 26 | 1.4 | 0.55 |
| | (23) | (15) | (18) | (0.24) | (0.21-1.40) |
| Swallowing | 2 | 1 | 3 | 0.19 | 0.30 |
| | (4) | (1) | (2) | (0.67) | (0.005-5.90) |
| Missing N | 2 | 1 | | | |

^{#}Combination refers to patients consuming MC inflorescence concomitantly with MC oil extract;
*Administration modes do not add to 100% due to concomitant modes; CI, Confidence interval; IQR, Inter quartile range; BMI, Body mass index; MC, Medical cannabis.

### Migraine and Sleep Features

Samples were divided into non-responders (i.e., <50%; n=56, 39%) and responders (i.e., >50%; n=89, 61%) based on their reduction of monthly migraine attacks frequency from pre-MC to current post-MC period. No significant difference was found in monthly migraine attacks frequency prior to MC treatment initiation [15 (7.8-30) and 14 (8-27), respectively)] [0.06 95% CI (−0.27 to 0.41); p=0.71], strengthening the division methodology, as both subgroups started from a similar standpoint. Moreover, there were no significant differences between the subgroups in any of the current migraine features, including the age of migraine diagnosis, average duration of migraine attacks, activity induced aggravation of migraine, unilateral migraine, bilateral migraine, presence of aura prior to migraine, nausea during migraine and/or phono/photophobia during migraine (Table 2).

TABLE 2
Migraine features.

| | | | | |
|---|---|---|---|---|
| | Non- | | | |
| | responders | Responders | Statistic | Effect size |
| Measure | N = 56 | N = 89 | (p) | (CI) |

Median (IQR)

| | | | | |
|---|---|---|---|---|
| Age of migraine | 20 | 22 | 0.07 | 0.07 |
| diagnosis (years) | (14-36) | (14-32) | (0.98) | (−0.27-0.42) |
| Missing N | 1 | 4 | | |
| Average migraine | 20 | 15 | 0.12 | 0.15 |
| duration (hours) | (5.8-35) | (5-48) | (0.72) | (−0.19-0.49) |
| Missing N | 1 | 2 | | |

Number of patients (%)

| | | | | |
|---|---|---|---|---|
| Activity induced | 32 | 61 | 1.20 | 1.60 |
| aggravation of | (57) | (69) | (0.28) | (0.73-3.3) |
| migraine | | | | |
| Missing N | 1 | 0 | | |
| Unilateral | 40 | 59 | 0.39 | 0.74 |
| migraine | (71) | (66) | (0.53) | (0.33-1.60) |
| Missing N | 1 | 0 | | |
| Aura+ | 16 | 31 | 0.28 | 1.30 |
| | (29) | (35) | (0.60) | (0.60-2.9) |
| Missing N | 1 | 0 | | |
| Nausea+ | 25 | 51 | 1.50 | 1.60 |
| | (45) | (57) | (0.23) | (0.78-3.40) |
| Missing N | 1 | 0 | | |
| Phono/photo | 38 | 60 | 0.00 | 0.93 |
| phobia+ | (68) | (67) | (0.98) | (0.42-2.00) |
| Missing N | 1 | 0 | | |

CI, Confidence interval; IQR, Inter quartile range, +, positive for this manifestation.

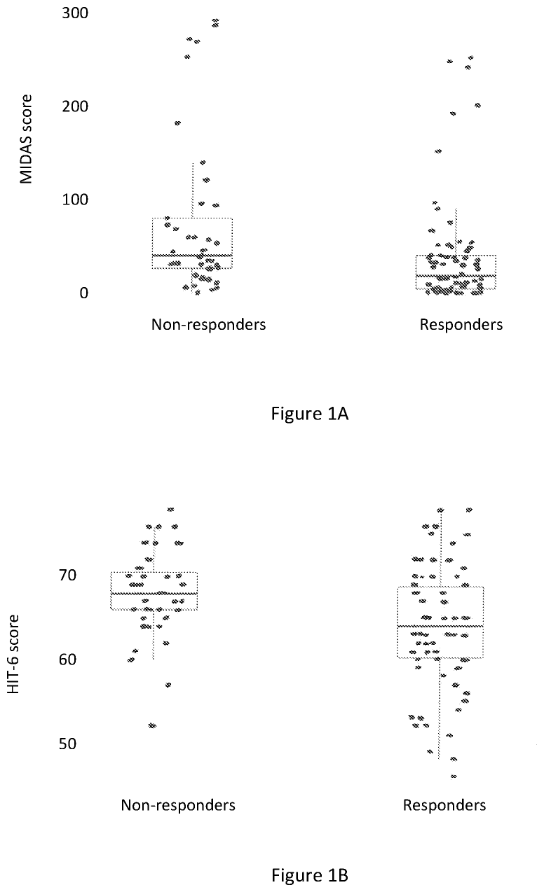

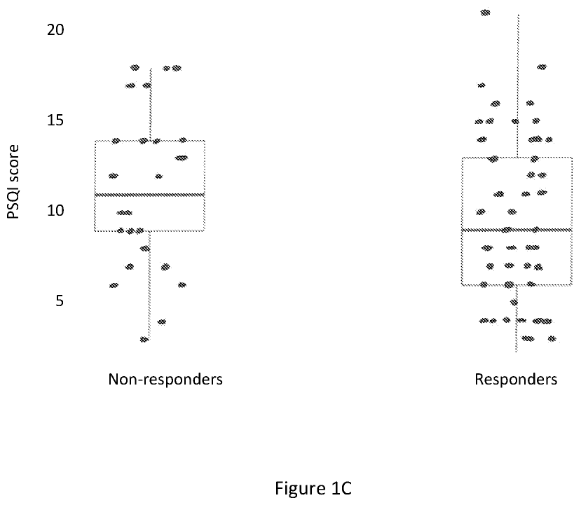

Responders were found to be more likely to report lower MIDAS (FIG. 1A) and HIT-6 (FIG. 1B) questionnaires scores [18 (5-40) and 64 (60-69), respectively] than non-responders [40 (26-80) and 68 (66-70), respectively] [0.50 95% CI (0.11 to 0.90); p<0.05 and 0.66 95% CI (0.26 to 1.00); p<0.001, respectively]. Moreover, responders reported better sleep quality [9 (6-13)] than non-responders [11 (9-14)] [0.46 95% CI (0.03 to 0.89); p<0.05] (FIG. 1C). Nonetheless, the evaluated sleep timing measures of sleep latency and sleep duration did not vary significantly between the migraine response subgroups (Table 3).

TABLE 3
Sleep characteristics.

| | | | | |
|---|---|---|---|---|
| | Non- | | | |
| | responders | Responders | Statistic | Effect size |
| Measure | N = 56 | N = 89 | (p) | (CI) |

Median (IQR)

| | | | | |
|---|---|---|---|---|
| Sleep quality | 11 | 9 | 0.30 | 0.46 |
| global score | (9-14) | (6-13) | (0.04) | (0.03-0.89) |
| (PSQI, 0-21) | | | | |
| Missing N | 22 | 27 | | |
| Sleep latency | 32 | 30 | 0.09 | −0.07 |
| (minutes) | (20-60) | (15-60) | (0.97) | (−0.46-0.33) |
| Missing N | 16 | 21 | | |
| Sleep duration | 6.2 | 6 | 0.11 | −0.09 |
| (hours) | (5-7) | (5-7) | (0.92) | (−0.49-0.30) |
| Missing N | 16 | 20 | | |
| Subjective | 3 | 2.5 | 0.18 | 0.42 |
| sleep quality* | (2-3) | (1-3) | (0.39) | (0.02-0.81) |
| Missing N | 15 | 19 | | |
| Sleep latency* | 2 | 2 | 0.15 | 0.2 |
| | (1.8-3) | (1-3) | (0.65) | (−0.20-0.59) |
| Missing N | 16 | 21 | | |
| Sleep duration* | 1 | 1 | 0.1 | −0.02 |
| | (0-2) | (0-2) | (0.95) | (−0.41-0.37) |
| Missing N | 16 | 20 | | |
| Habitual sleep | 1 | 0 | 0.09 | 0.08 |
| efficiency* | (0-2) | (0-2) | (0.99) | (−0.32-0.49) |
| Missing N | 18 | 22 | | |
| Sleep | 2 | 2 | 0.19 | 0.59 |
| disturbances* | (2-2) | (1-2) | (0.33) | (0.19-0.98) |
| Missing N | 15 | 19 | | |
| Use of sleeping | 1 | 0 | 0.19 | 0.35 |
| medication* | (0-3) | (0-1.2) | (0.34) | (−0.05-0.75) |
| Missing N | 17 | 21 | | |
| Daytime | 2 | 1 | 0.18 | 0.34 |
| dysfunction* | (1-2) | (1-2) | (0.40) | (−0.06-0.74) |
| Missing N | 17 | 23 | | |

*Components of the PSQI questionnaire global score; CI, Confidence interval; IQR, Inter quartile range; PSQI, Pittsburgh sleep quality index.

### MC Treatment Safety

MC related adverse effects (AEs) were reported by 37% (n=53) of the sample. Notably, non-responders reported higher incidences of any AEs (n=26, 46%) than responders (n=27, 30%) [0.46 95% CI (0.21 to 0.99), p<0.05]. Most of the specific AEs did not vary significantly between responders and non-responders. However, itchy and red eyes (n=8, 9%, for both) were reported only in the responders' subgroup (χ²(1)=6.9, p<0.01, for both). Additionally, dry mouth was reported at higher rates among the responders (n=9, 10%) than among the non-responders (n=2, 4%) (χ²(1)=3.9, p<0.05).

In descending order of frequency, AEs report included central nervous system AEs (n=33, 23%), psychological AEs (n=21, 14%), ophthalmic AEs (n=16, 11%), gastrointestinal AEs (n=15, 10%), musculoskeletal AEs (n=11, 8%), cardiovascular AEs (n=10, 7%), and auditory AEs (n=9, 6%).

There were no significant differences between patients reporting MC related AEs in different MC administration modes (i.e., inflorescence, oil extract or combination of these administration modes) [0.08 95% CI (0 to 0.25); p=0.59]. Additionally, no such differences were found in the specific administration modes of inflorescence and of oil extract (p>0.05).

### MC Treatment Complexity

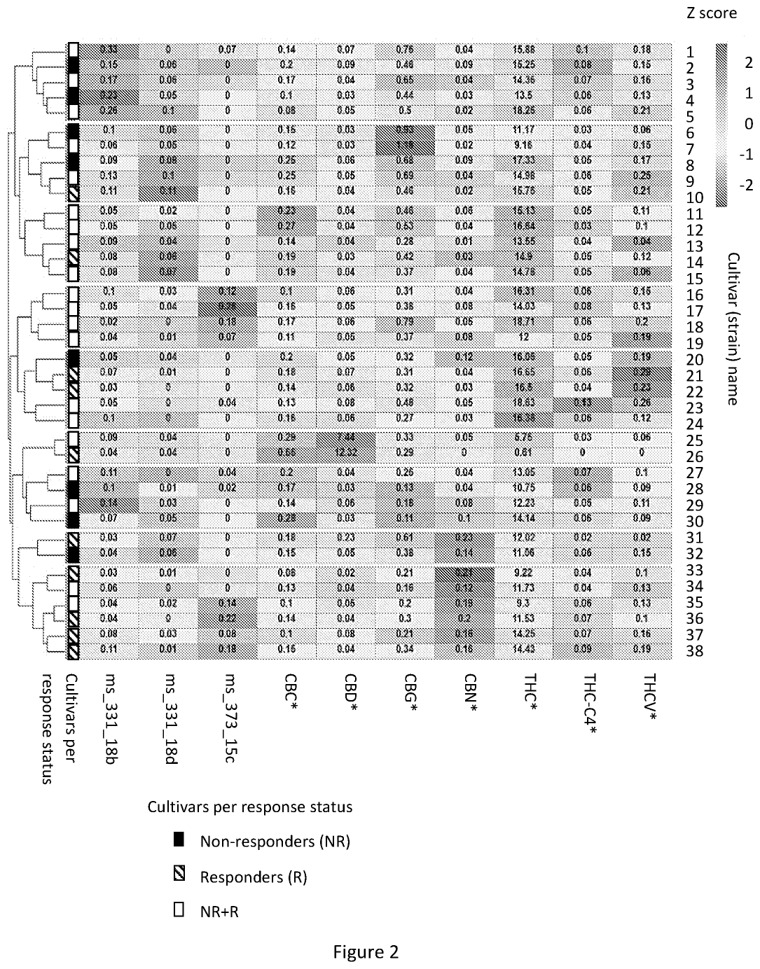

The complexity of MC treatment in Israel is due to the variety of available cultivars in Israel (about 100 different cultivars or “strain names”) and the options of patients to consume more than one cultivar in the same month with varying doses of each cultivar. Consequently, 50 unique MC cultivar combinations were reported in the current study by the 68 patients for which full cultivar/s lab information was available. Notably, 46 (92%) of the 50 possible combinations were THC dominant cultivars, three (6%) were combinations of CBD and THC dominant cultivars and one (2%) was of CBD dominant cultivars only. These 50 combinations were comprised of 38 unique cultivars. FIG. 2 shows the analysis of phytocannabinoids of these 38 cultivars consumed by the sample subgroup. Based on the phytocannabinoids concentration variability, these cultivars were clustered by Z score clustering to nine different clusters. FIG. 2 also shows that in the combinations of cultivars consumed, ten cultivars were consumed only by responders, eight cultivars were consumed only by non-responders and the rest of the cultivars (n=20) were consumed both by responders as well as by non-responders.

### MC Treatment Characteristics

In this subgroup analysis, only data on patients that smoked and/or vaped MC inflorescences and not of those who consumed oil extracts sublingually, were included in order to avoid comparing between different modes of administration (different pharmacodynamics). Since the inflorescences in this study were analyzed in their natural form, monthly consumption of phytocannabinoid doses were calculated using total phytocannabinoid concentrations, rather than analyzing separate acid and/or neutral concentrations, in order to simulate the neutral maximum content of phytocannabinoids consumed following smoking and/or vaporization. This calculation corrected for any differences that may have arisen in phytocannabinoid profiles as a result of decarboxylation due to mishandling or storage of the MC inflorescences. Thus, the minority of patients that reported sublingual consumption of oil extract/s (n=12) or a combination of sublingual consumption of oil extract/s with inflorescence/s (n=19) were not included in this subgroup analysis. Consequently, 68 (47%) patients reported on exclusive MC inflorescence/s consumption via inhalation. 45 (66%) of them were responders and 23 (34%) were non-responders.

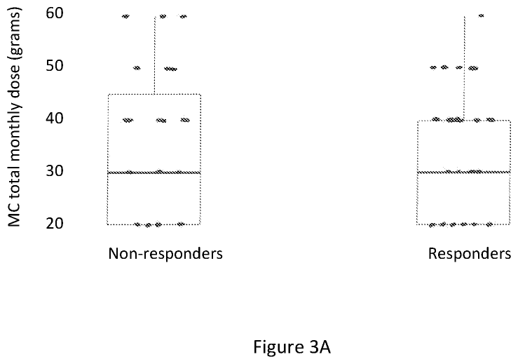

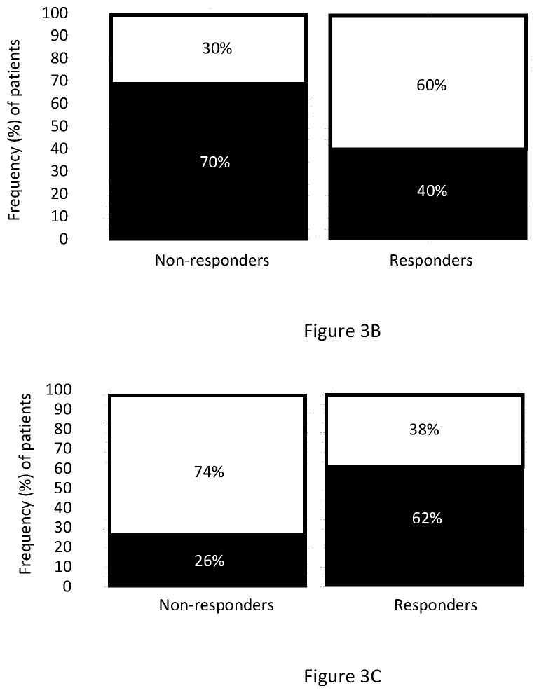

For the abovementioned 68 patients, the differences of total MC monthly dose between responders and non-responders was first evaluated. No significant difference was found [30 (20-40) grams and 30 (20-45) grams, respectively] [0.25 95% CI (−0.26 to 0.76); p=0.97] (FIG. 3A). The impact of the specific phytocannabinoids monthly dose was then evaluated. As the distribution of specific phytocannabinoids monthly doses were non-normal, specific phytocannabinoids were separated to low and high monthly dose groups, based on the distribution of consumption in the patients' sample.

Responders were found to be more likely to consume a high dose (7.9-109.5 mg per month) of the phytocannabinoid denoted CAN002 (n=27, 60%) and a low dose (0-9.9 mg per month) of the phytocannabinoid denoted CAN001 (n=28, 62%) compared to non-responders that were more likely to consume a low dose (0-7.8 mg per month) of CAN002 (n=16, 70%) and a high dose (10.0-46.8 mg per month) of CAN001 (n=17, 74%) [3.40 95% CI (1.10 to 12.00); p<0.05 and 0.22 95% CI (0.05 to 0.72); p<0.01, respectively]. (FIGS. 3B-3C). The other phytocannabinoids monthly doses did not vary significantly between the subgroups. Importantly, no differences were found between responders and non-responders in the daily frequency of MC consumption [5 (2.5-7) times per day and 4.5 (3-6) times per day, respectively] [0.18 95% CI (−0.34 to 0.71), p=0.99]. Additionally, no differences were found in the number of monthly cannabis cultivars combinations [2 (1-2) cultivars, respectively] [0.04 95% CI (−0.47 to 0.56), p=0.99]. Interestingly, of the 38 unique cultivars that patients consumed in their combinations, 12 cultivars contained considerable amount of CBGMA and none to very low amounts of CAN001. These cultivars appeared more frequently among the responders (42 appearances in cultivars combinations), than among the non-responders (14 appearances in cultivars combinations). All the cultivars with the aforementioned beneficial ratio of high CBGMA and low CAN001 monthly doses were THC-dominant cultivars and had less than one percent of CBD.

### Migraine Treatment Characteristics

A total of 65 (45%) of the patients reported any current use of pharmaceutical analgesic medications consumption. Although not significant [0.51 95% CI (0.23 to 1.10), p=0.09], more of the non-responders (n=30, 54%) consumed analgesics compared to responders (n=35, 39%). Nonetheless, non-responders consumed significantly higher rates of weak opioids (n=13, 23%; e.g., Tramadol Hydrochloride, Buprenorphine Hydrochloride, etc.), strong opioids (n=14, 25%; e.g., Oxycodone Hydrochloride, Fentanyl, etc.) and triptans (n=9, 16%; e.g., Sumatriptan, Rizatriptan, etc.) compared to responders (n=4, 5%; n=7, 8% and n=4, 5%, respectively) [0.15 95% CI (0.03 to 0.53); p<0.005, 0.25 95% CI (0.07 to 0.72); p<0.005 and 0.24 95% CI (0.05 to 0.93), p<0.05]. No statistically significant variations were found between responders and non-responders in the consumption rates of over the counter (OTC) analgesics, NSAIDs, anticonvulsants, antidepressants, and antiemetics.

In this cross-sectional study, patient reports on the frequency of their monthly migraine attacks, both pre-MC treatment and post-MC treatment, were evaluated. Patients were classified as responders if they reported more than 50% reduction in monthly migraine attacks post-MC treatment. Responders reported lower current migraine disability and lower negative impact compared to non-responders. In particular, responders reported better migraine disability status, less negative headache impact and better sleep quality. These findings suggest that improved migraine disability status and negative impact among MC treatment responders is attributed directly to MC treatment effects, and not secondary to the reduction of the frequency of migraine attacks. An association between patients with poor sleep quality and less responsiveness to MC treatment in reducing the frequency of migraine attacks is also reported.

The results show that responders to MC treatment also reported lower rates of opioids and triptans consumption compared to non-responders. Without being bound by any theory or mechanism of action, it is believed that these results indicate that patients that responded clinically to MC treatment, substituted this conventional treatment for MC. The incidence of MC related AEs were higher among non-responders. Without being bound by any theory or mechanism of action, this finding might be because responders tolerate MC related AEs better than non-responders or because non-responders trial and error choice of MC chemovar led to a different consumption of MC components' composition that led to a higher rate of AEs.

The differences in relative monthly dose of cannabinoids in each cultivar/s consumed, in both the responders and non-responders groups were evaluated. This provided the assessment of the dose consumption of wide variety of specific phytocannabinoids administered in combinations of cultivars. By doing so, elucidation of associations between specific cannabinoids consumed over a monthly dose, and the clinical response of migraine frequency reduction since MC treatment initiation is provided. Higher rates of patients that reported significant migraine frequency reduction following MC treatment were found to consume higher monthly doses of CAN002 and lower monthly doses of CAN001. These compounds were identified in both Δ⁹-THC- and CBD-rich chemovars according to LC/MS/MS. According to their MS/MS fragmentation spectra, CAN002 and CAN001 are acid and neutral phytocannabinoids, respectively.

This study has few limitations. First, the small sample size might have biased the results. Nonetheless, non-parametric models were used. Second, self-report bias could have occurred. However, the questionnaire was anonymous and validated, and patients answered with no effect on their current treatment by their physician. Lastly, since frequency of migraine attacks prior to MC treatment was reported in retrospect, recall bias might have occurred.

In conclusion, specific cultivars that contain the favorable ratio of compounds that were associated with migraine frequency reduction were identified. These specific cultivars were shown to induce long-term reduction of migraine frequency in over 60% of treated patients which was further associated with less disability and lower anti-migraine medications intake. These specific cultivars and pharmaceutical compositions comprising these cannabinoid compounds are therefore contemplated as being effective in the treatment of migraines.

### Example 2

In order to test the efficacy of CAN001 and CB GM in the treatment of migraines, a cAMP accumulation assay (LANCE Ultra cAMP assay kit by PerkinElmer, Inc.) utilizing the human neuroblastoma cells SK-N-MC that endogenously express human CGRP receptors is used. Cells are cultured in minimum essential medium (MEM) containing 10% fetal bovine serum (FBS) and 1% glutamine/penicillin. CAN001 and/or CBGM at a concentration of 0.2 μg/ml to 2 μg/ml are tested by incubation with SK-N-MC cells plated in 96-well half-area white plates (2000 cells/well) for 15 minutes at room temperature. CGRP (0.3 nM final concentration, EC80) is then added and further incubated for 15 minutes at room temperature. Following an additional 60-minute incubation at room temperature, the assay plates are read on CLARIOStar Plus™ instrument (BMG LABTECH) at an emission wavelength of 665 nm and the data are analyzed to calculate IC₅₀ values using GraphPad Prism software version 5.1 (GraphPad Software Inc.).

### Example 3

In order to test the efficacy of CAN001 and CB GM in the treatment of migraines, a cAMP accumulation assay and Ca²⁺ flux are measured utilizing differentiated neuronal progenitor cells. ReNcell VM is an immortalized human neural progenitor cell line with the ability to differentiate into neurons and glial cells. ReNcell VM is derived from the ventral mesencephalon region of human fetal brain and it retains a normal diploid karyotype in culture even after prolonged passage. This line can differentiate into neurons, astrocytes, and oligodendrocytes, thus creating a neuronal cell system. CALCRL (CGRP receptor) is expressed in the progenitor cells and upregulated in differentiation cultures (Pai et al. J Biomol Screen 2012, 17, 1180). RenCell VM are grown and differentiated by growth factor deprivation as described in Kim et al. (Nature Protocols. 2015, 10(7), 985-1006). The cells are differentiated into neurons, astrocytes, and oligodendrocytes, thus creating an active and mature neuronal cell system. The culture is tested for mature neuronal markers, neuronal activity and expression of CGRP receptors as described in Kim et al. (Nature Protocols 2015, 10(7), 985-1006) and Pai et al. (J Biomol Screen 2012, 17, 1180). The cultures are then treated with CGRP in combination with CAN001 and/or CBGM at a concentration of 0.2 μg/ml to 2 μg/ml. Both cAMP and Ca²⁺ levels post treatment are measured.

### Example 4

In order to test the efficacy of CAN001 and CBGM in the treatment of migraines, light aversion and motility assay in nestin/hRAMP1 mice harboring the human gene for CGRP receptor are utilized. CGRP-induced light-aversive behavior is used in mice as a measure of migraine-associated photophobia. Light/dark boxes with infrared beam tracking (Med Associates) for light aversion and motility assay as described in Kaiser et al. (J Neurosci. 2012, 32(44), 15439-15449) and Mason et al. (J Neurosci. 2017, 37(1), 204-216) are utilized. Mice are administered with CGRP in combination with CAN001 and/or CBGM at a concentration of 5 mg/kg to 50 mg/kg body weight via intracerebroventricular injection in the right lateral ventricle as described in Recober et al. (J Neurosci. 2009, 29(27), 8798-8804). Following administration, mice are allowed to recover for 60 min in their home cages before testing.

Wild-type mice are pre-exposed to the chamber twice every third day before treatment exposure. After exposure, mice are tested in the light/dark boxes 3 days after treatment. In addition, mice are tested using bright light (27,000 lux; naive), and dim light (55 lux; transgenic) as described in Kaiser et al. (J Neurosci. 2012, 32(44), 15439-15449). Data are collected for 30 minutes and analyzed in 5 minutes intervals. The average time spent on each side of the chamber per 5 minutes interval is also measured.

Open-field assay is performed as described in Kaiser et al. (J Neurosci. 2012, 32(44), 15439-15449). Mice are placed in the center of the chamber and tested for 30 min. The periphery is defined as 4.22 cm from the border with the remaining 18.56×18.56 cm area as the center. Motility outcomes are measured as described in Kaiser et al. (J Neurosci. 2012, 32(44), 15439-15449). To account for the variation in the amount of time mice spend in each zone, data are normalized to time spent in the dark and light zones.

### Example 5

In order to test the efficacy of CAN001 and CBGM in the treatment of migraines, cAMP and Ca²⁺ accumulation assays employing human iPSC-derived trigeminal neurons are utilized. Human iPSCs of migraine patients from an iPSC collection (Public Health England, culture collection) are obtained. Healthy controls and migraine patient's iPSCs are differentiated to TG neurons as described in Bastian Zimmer et al., 2018 (PNAS 2018, 115(37), E8775-E8782). Differentiation is followed by functional characterization of trigeminal neurons to verify expression of trigeminal markers and CGRP receptors by performing functional receptor assays. The cultures are then incubated with CGRP in combination with CAN001 and/or CBGM at a concentration of 0.2 μg/ml to 2 μg/ml and the effect of cAMP accumulation and Ca²⁺ flux is measured as described in Pai et al. (J Biomol Screen 2012, 17, 1180).

It will be appreciated by persons skilled in the art that the present invention is not limited by what has been particularly shown and described herein above. Rather, the scope of the invention is defined by the claims that follow.

1-24. (canceled)

25. A method of treating or preventing a headache, the method comprising administering to a subject in need thereof a pharmaceutical composition comprising at least one cannabinoid selected from CAN001, CBGM, and pharmaceutically acceptable salts or derivatives thereof.

26. The method of claim 25, wherein the headache is a migraine headache.

27. The method of claim 25, wherein the pharmaceutical composition comprises a combination of CAN001 and CBGM or pharmaceutically acceptable salts or derivatives thereof.

28. The method of claim 27, wherein the weight percent ratio of CAN001 and CBGM or pharmaceutically acceptable salts or derivatives thereof is in the range of from 2:1 to 1:100.

29. The method of claim 28, wherein the weight percent ratio of CAN001 and CBGM or pharmaceutically acceptable salts or derivatives thereof is in the range of from 1:2 to 1:100.

30. The method of claim 25, wherein the pharmaceutical composition further comprises at least one additional cannabinoid selected from the group consisting of tetrahydrocannabinol (THC), THC-C4, tetrahydrocannabivarin (THCV), cannabinol (CBN), cannabigerol (CBG), cannabidiol (CBD), cannabichromene (CBC), and a mixture or combination thereof.

31. The method of claim 25, wherein the pharmaceutical composition further comprises at least one additional cannabinoid selected from the group consisting of tetrahydrocannabinol (THC), THC-C4, tetrahydrocannabivarin (THCV), cannabinol (CBN), cannabigerol (CBG), cannabidiol (CBD), cannabichromene (CBC), cannabidivarinic acid (CBDVA), cannabidiolic acid (CBDA), cannabigerolic acid (CBGA), cannabinolic acid (CBNA), cannabichromenic acid (CBCA), tetrahydrocannabinolic acid (THCA), THCA-C4, cannabicitran, sesquicannabigerol (sesqui-CBG), sesquicannabigerolic acid (sesqui-CBGA), CBGA-C4, CBG-C4, cannabigerovarinic acid (CBGVA), cannabigerivarin (CBGV), cannabigerorcinic acid (CBGOA), cannabigerorcin (CBGO), cannabicyclol (CBL), cannabicyclolic acid (CBLA), tetrahydrocannabivarin carboxylic acid (THCVA), tetrahydrocannabiorcolic acid (THCOA), tetrahydrocannabiorcol (THCO), THCMA, THCM, CBDA-C4, CBD-C4, cannabidiorcolic acid (CBDOA), cannabidiorcol (CBDO), cannabidiolic acid monomethyl ether (CBDMA), cannabidiol monomethylether (CBDM), cannabichromenic acid (CBCA), cannabichromene (CBC), cannabichromevarinic acid (CBCVA), cannabichromevarin (CBCV), cannabiorchromenic acid (CBCOA), cannabiorchromene (CBCO), cannabinolic acid (CBNA), cannabinol-C4 (CBN-C4), cannabivarinic acid (CBNVA), cannabivarin (CBNV), cannabiorcolic acid (CBNOA), cannabiorcol (CBNO), CBNA-8-OH, CBN-8-OH, cannabinol methylether (CBNM), cannabielsoin acid (CBEA), cannabielsoin (CBE), cannabielsoic acid (CBEVA), cannabielsoin (CBEV), cannabinodiolic acid (CBNDA), cannabinodiol (CBND), cannabinodivarinic acid (CBNDVA), (−)-Δ⁸-trans-tetrahydrocannabinol (Δ⁸-THC), cannabitriol-1 (CBT-1), CBT-2, CBT-3, CBTA-1, CBTA-3, cannabitriolvarin (CBTV), CBTV-3, and a mixture or combination thereof.

32. The method of claim 30, wherein the pharmaceutical composition comprises THC, CAN001, and CBGM, and pharmaceutically acceptable salts or derivatives thereof.

33. The method of claim 32, wherein the weight percent ratio of THC, CAN001 and CBGM or pharmaceutically acceptable salts or derivatives thereof is in the range of from 2,000:2:1 to 50:1:100.

34. The method of claim 25, wherein the pharmaceutical composition comprises a single cannabinoid.

35. The method of claim 34, wherein the cannabinoid is CAN001 or pharmaceutically acceptable salts or derivatives thereof.

36. The method of claim 34, wherein the cannabinoid is CBGM or pharmaceutically acceptable salts or derivatives thereof.

37. The method of claim 25, wherein the pharmaceutical composition comprises CAN001 or pharmaceutically acceptable salts or derivatives thereof and at least one additional cannabinoid.

38. The method of claim 25, wherein the pharmaceutical composition comprises CBGMA or pharmaceutically acceptable salts or derivatives thereof and at least one additional cannabinoid.

39. The method of claim 25, wherein the cannabinoid is a natural compound, a synthetic compound, a semi-synthetic compound, or a mixture thereof.

40. The method of claim 25, wherein the pharmaceutical composition further comprises at least one pharmaceutically acceptable excipient.

41. The method of claim 40, wherein the at least one pharmaceutically acceptable excipient is selected from the group consisting of a binder, a filler, a surfactant, an anti-tacking agent, a plasticizer, a lubricant, a glidant, a disintegrant, a diluent, a tonicity enhancing agent, a wetting agent, a mucoadhesive agent, a buffering substance, a colorant, a preservative, and any combination thereof.

42. The method of claim 25, wherein the pharmaceutical composition is in a dosage form selected from tablet, pill, capsule, strip, pellets, granules, powder, lozenge, sachet, cachet, elixir, suspension, dispersion, emulsion, solution, syrup, aerosol, foam, gel, ointment, lotion, cream, and suppository.

43. The method of claim 25, wherein administration is performed via a route selected from oral, buccal, sublingual, subcutaneous, intratracheal, intrabronchial, intra-alveolar, intraperitoneal, rectal, intravenous, intra-arterial, transdermal, intramuscular, topical, and intranasal.

44. The method of claim 25, wherein the subject is a mammal.
